(19) United States
(12) Patent Application Publication
(10) Pub. No.: US 2005/0137523 A1
(43) Pub. Date: Jun. 23, 2005

(54) Medicament administration apparatus

(76) Inventors: Philip W. Wyatt, Glendale, CA (US); Gary L. Schaeffer, Glendale, CA (US)

Correspondence Address: James E. Brunton, Esquire, Post Office Box 29000, Glendale, CA, 91209-9000, US

(21) Appl. No.: 10/744,806

(22) Filed: Dec. 22, 2003

(51) Int. Cl. A61M 37/00

(52) U.S. Cl. 604/87

(57) ABSTRACT

An apparatus for removal of premixed drugs or reconstitution of lyophilized drugs and for the injection of the reconstituted drug into the patient. The apparatus includes a syringe assembly and an adapter assembly that can be removably connected to a medicament container containing a premixed drug or lyophilized medicament. The syringe assembly of the apparatus includes a body portion to form a liquid chamber between the forward end of the body portion and the piston and a syringe cannula assembly. The syringe cannula assembly, which can be removably interconnected with the body portion, comprises a cannula support and a hypodermic needle sealably connected to the cannula support. The adapter assembly comprises an adapter preferably molded from a moldable plastic that includes a top wall, an adapter cannula connected to and extending from the top wall and a variety of connectors connected to the top wall for removably interconnecting the adapter with the medicament container. The adapter assembly further includes syringe connector member connected to the top wall for removably interconnecting the syringe with the adapter in a manner to uniquely position the syringe cannula within the lumen of the adapter cannula wherein it is completely shielded from external contamination and prevent print damage and injury to the user.

### BACKGROUND OF THE INVENTION

1. Field of the Invention

The present invention relates generally to medicament administration. More particularly, the invention concerns a novel fluid medicament delivery apparatus that is specially designed to facilitate the aseptic administration of drugs to patients.

2. Discussion of the Prior Art

Medicaments intended for parenteral administration are typically stored in a medicament container in either in liquid, powdered or lyophilized form. Typically, lyophilized drugs are packaged in standard glass vials that are sealed with a pierceable rubber stopper and a crimped metal cap. A suitable liquid diluent must be added to the vial to reconstitute the powdered or lyophilized drug before use. In accordance with typical prior art methods, this reconstitution step was accomplished by drawing a measured amount of diluent, such as water, into a syringe from a diluent vial. The sealed vial containing the powdered or lyophilized drug was then accessed using a hypodermic needle and syringe to add the liquid diluent to the vial. The vial was then in inverted and shaken to intermix the drug with the liquid diluent. This done, the reconstituted drug was withdrawn into the syringe and was injected into the patient.

It is apparent that when reconstitution of a drug is required, the prior art processes required at least two fluid transfers. The problem of ensuring proper fluid transfer under acceptable aseptic conditions during these two fluid transfers was formidable and was especially acute in the case of self-administration of drugs by patients in a homecare environment. For example, during the fluid transfers, the rubber stopper that is disposed within the top of the vial must be penetrated by the syringe needle. Typically, the rubber stopper is not sterile and, accordingly, the exposed needle is exposed to non-sterile conditions. Furthermore, as the needle penetrates the rubber stopper it will inevitably become contaminated with small particles of rubber that are dislodged from the rubber stopper during the needle penetration step. Additionally, this two-step process is quite cumbersome for physicians and particularly for homecare caregivers to accomplish, often under the stressful conditions that frequently exist at the bedside of a patient.

One approach to overcoming the drawbacks of the prior art methods as described in the preceding paragraphs is disclosed in U.S. Pat. No. 6,238,372 to issued to Zinger et al. The Zinger et al. patent discloses a drug vial mixing and transfer device having one or more ports with interconnecting fluid passageways. The ends of the ports are attached either to a piercing connector or a syringe. The piercing connector is used to support and penetrate the rubber stoppers of the standard glass drug vials filled that are with powdered or lyophilized drugs or a liquid diluent, during the transfer of the liquid diluent and drug solutions between the vials and the syringe. In one form of the invention, the ports and connectors are mounted on a base and a stopcock type valve is used to coordinate communication between the fluid passageways of the different ports. Retainers mounted on the base hold the syringe and vials in place during the liquid transfer operations.

### SUMMARY OF THE INVENTION

By way of summary, the present invention concerns a disposable shrouded vial adapter with preconnected, integral “med push” hypodermic needle for low-cost, economical reconstitution of lyophilized drugs and for the direct injection of the reconstituted drug into the patient. In one form of the invention, the apparatus comprises a syringe assembly and a novel adapter assembly that can be removably connected to a medicament container or vial containing a liquid medicament, a powdered medicament or a lyophilized medicament. The syringe assembly of the apparatus includes an aspirator component that includes a body portion having a forward end and a piston slidably carried within the body portion to form a liquid chamber between the forward end of the body portion and the piston. The aspirator connector component of the syringe assembly, which comprises a syringe cannula assembly is adapted to be removably interconnected with the aspirator component. This novel aspirator connector component comprises a cannula support and a syringe cannula connected to the cannula support.

In one form of the invention the adapter assembly comprises an adapter, preferably molded from a moldable plastic, that includes a body portion having a tapered bore, a top wall connected to the body portion, an adapter cannula connected to and extending from the body portion and a container connector means connected to the top wall for removably interconnecting the adapter with the medicament container. The container connector means can be of various configurations that telescopically receive and securely grip the upper portion of the medicament container. Uniquely, when the cannula support portion of the aspirator connector component is sealably received within the tapered bore of the body portion of the adapter assembly, the syringe cannula portion of the aspirator connector component is strategically positioned within the lumen of the adapter cannula where it is completely shielded from external contamination.

It is an object of the present invention to provide a method and apparatus for reconstituting a lyophilized drug and for then delivering the reconstituted drug to a patient The method of the invention makes use of an apparatus of the character described in the preceding paragraph and is carried out in a manner such that the hypodermic syringe component of the apparatus is at all times protected from external contaminants and need not be used to penetrate the rubber stopper of the medicament container containing the drug that is to be reconstituted.

Another object of the invention is to provide a method of the aforementioned character in which off-the-shelf syringe body components that have been pre-filled with a suitable diluent can be used to accomplish the reconstitution step of the method of the invention.

Another object of the invention is to provide apparatus of the class described in which the adapter component includes filter means for filtering the fluid that is aspirated from the medicament container.

Another object of the invention is to provide apparatus of the class described in which the adapter component includes vent means for venting to atmosphere any gases that may be contained within the medicament container.

Another object of the invention is to provide apparatus of the class described herein that is of a simple design and is easy use in both hospital and homecare environments.

Another object of the invention is to provide an apparatus as described in the preceding paragraph which can be inexpensively manufactured so that the apparatus can be economically disposed of after use.

Another object of the invention is to provide apparatus of the class described herein that can conveniently be used to reconstitute and deliver a wide variety of medicaments in various selected doses.

### BRIEF DESCRIPTION OF THE DRAWINGS

FIG. 1 is a side elevational view, partly in cross-section showing one form of the vial accessing adapter subassembly of the invention for use in administering medicaments to a patient.

FIG. 2 is a view is similar to FIG. 1, but showing the vial accessing adapter subassembly positioned over and interconnected with a conventional medicament container or vial that is shown in phantom.

FIG. 2A is an enlarged view similar to FIG. 2, but illustrating in cross-section the area designated in FIG. 2 as 2A.

FIG. 3 is a view partly in cross section of the accessing adapter subassembly and the syringe body that can be operably interconnected with the accessing adapter subassembly to form the syringe assembly that is used to accomplish the fluid transfer steps.

FIG. 3A is a view similar to FIG. 3, but showing the syringe assembly separated from the adapter component of the accessing adapter assembly.

FIG. 4 is a fragmentary cross-sectional view of an alternate form of the vial accessing adapter subassembly of the apparatus of the invention that includes filter means for filtering the fluid withdrawn from the medicament container.

FIG. 4A is a greatly enlarged, cross-sectional view taken along lines 4A-4A of FIG. 4.

FIG. 5 is a cross-sectional view of another form of the adapter component of the vial accessing adapter means of the invention that includes venting means for venting to atmosphere gases contained within the medicament container.

FIG. 5A is an enlarged, cross-sectional view of the area designated in FIG. 5 as 5A.

FIG. 6 is a cross-sectional view of still another form of the vial accessing adapter means of the invention having an adapter component of a different configuration.

FIG. 7 is a cross-sectional view of yet another form of the vial accessing adapter means of the invention having an adapter component that includes a circumferentially extending, neck-gripping bead.

FIG. 8 is a cross-sectional view of yet another form of vial accessing adapter means for accessing a medicament container.

### DESCRIPTION OF THE INVENTION

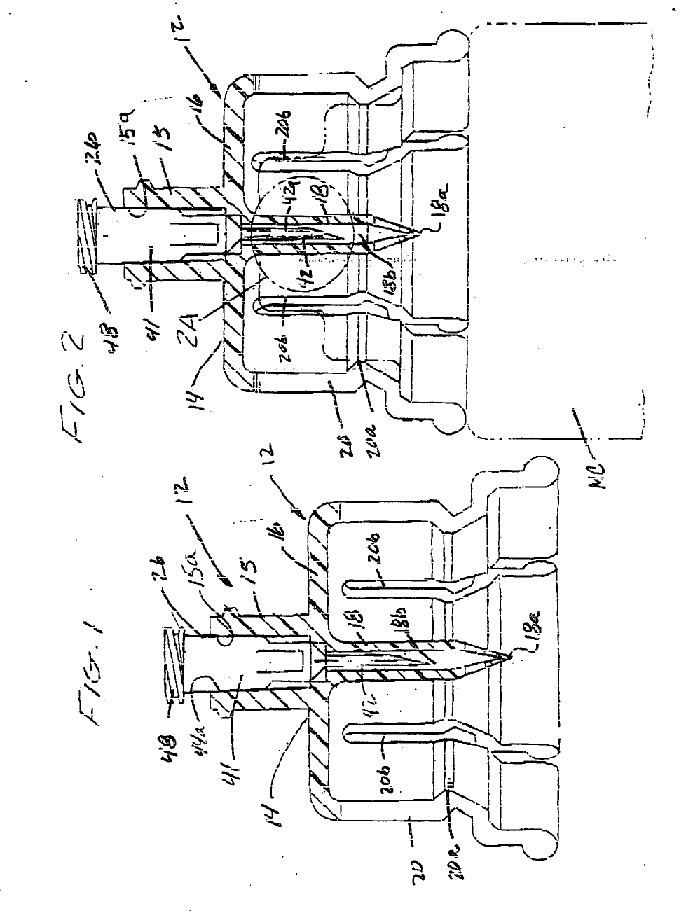

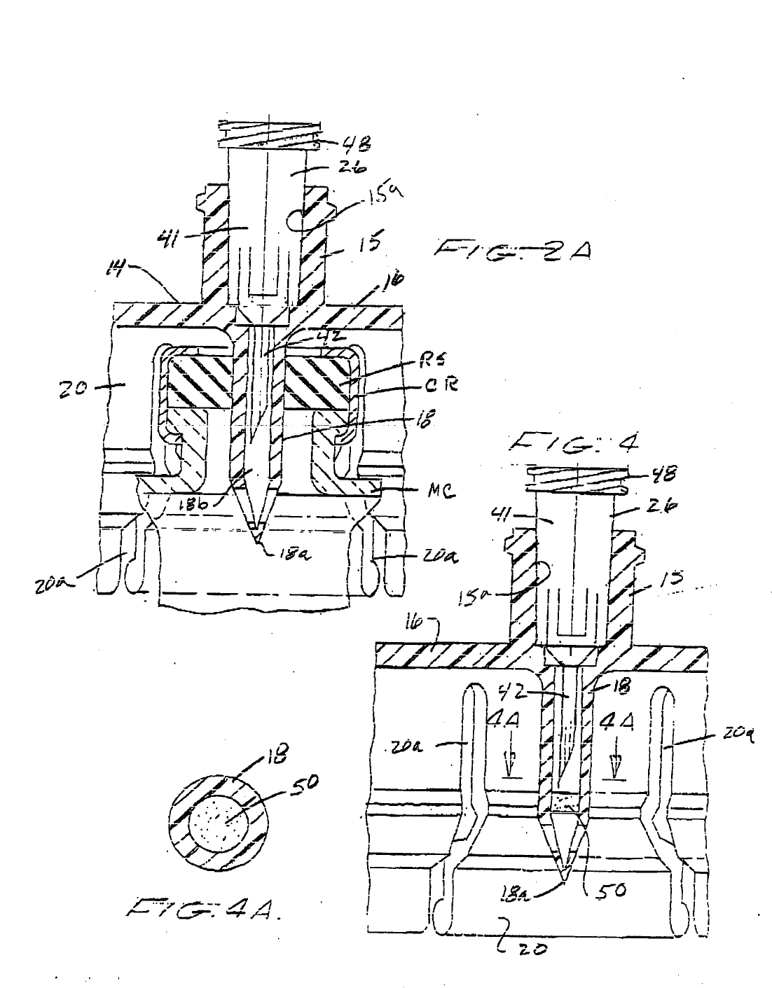

Referring to the drawings and particularly to FIGS. 1 and 2, one form of the vial accessing adapter means of the invention for the aseptic administration of medicaments contained within a medicament container is there shown. The vial-accessing adapter means here comprises a vial accessing adapter subassembly 12 that includes an adapter component 14 that is preferably formed from a moldable plastic material. Adapter component 14 includes a body portion 15 having a tapered bore 15a, a top wall 16 connected to the body portion and an adapter cannula 18 that is integrally formed with and extends from body portion 15. A container connector means is also connected to top wall 16 and functions to removably interconnect the adapter component with a conventional medicament container MC (FIG. 2). The container connector means of the present form of the invention here comprises a resiliently deformable skirt 20 that is integrally formed with and extends from top wall 16 in the manner shown in the drawings. As best seen in FIG. 2, when the adapter component is mated with the medicament container, skirt 20 telescopically receives and securely grips the upper portion of the medicament container in a manner such that adapter cannula 18 will completely pierce the rubber stopper RS of the container (FIG. 2A). Rubber stopper RS is secured in position within the upper portion of the container in a conventional manner by a crimp ring CR.

As illustrated in FIG. 2A, plastic adapter cannula 18 has a piercing extremity 18a and a lumen 18b that communicates with the interior of the medicament container MC when the adapter component is interconnected with the container in the manner shown in FIG. 2A. In this regard, it is to be noted that skirt 20 is provided with a radially inwardly extending shoulder 20a and a plurality of circumferentially spaced slits 20b that enable the adapter component of the invention to be snapped over the upper portion of the medicament container MC to securely grip the container in the manner shown in FIG. 2.

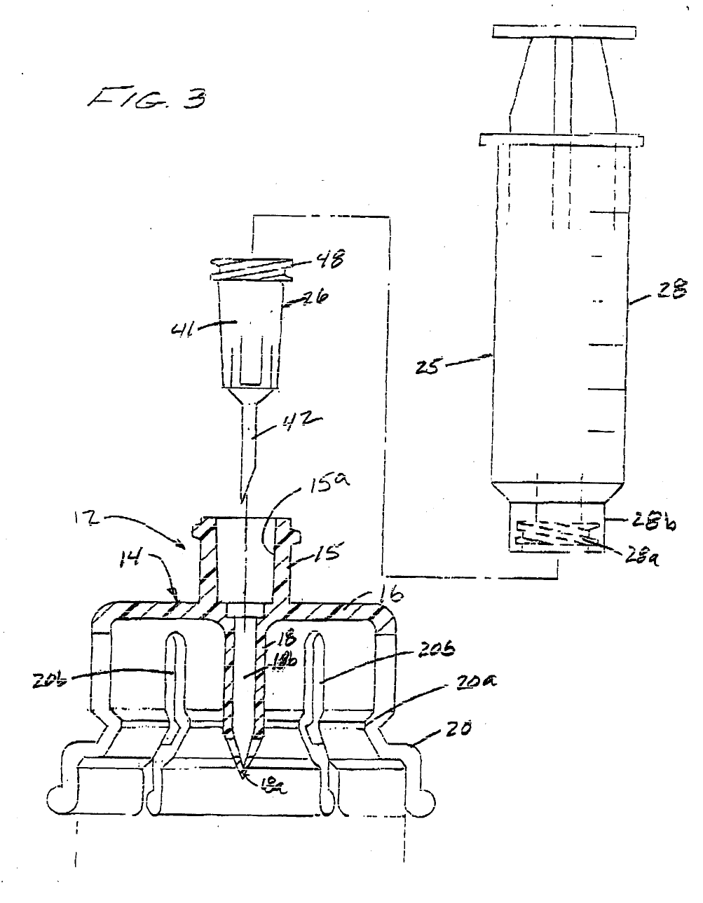

In the present form of the invention body portion 15 of the vial accessing adapter means functions to interconnect the syringe assembly 24 (FIG. 3A) of the apparatus of the invention with the-medicament container MC. As illustrated in FIG. 3, this important syringe assembly 24 is made up of an aspirator means shown here as syringe body 25 and an aspirator connector means, or syringe cannula assembly 26, that can be sealably connected to body portion 15 of the vial accessing adapter means of the invention. The aspirator means or syringe body 25 includes a barrel portion 28 having a forward end 28a and a piston 30 that it is slidably carried within the barrel portion. As in conventional syringes, a liquid chamber 32 is formed between the forward end 28a of the barrel portion of the syringe body and piston 30.

Removably connected to the aspirator means or syringe body 25 is the previously mentioned aspirator connector means or syringe cannula assembly 26 that includes a syringe cannula support 41 and a syringe cannula 42 that is integrally formed with the syringe cannula support 41. As best seen in FIG. 2, syringe or aspirator cannula 42 is here shown as a hypodermic needle having a lumen 42a. As shown in FIG. 3, syringe cannula assembly 26, which is removably connected to syringe body portion 25 of the syringe assembly, is sealably receivable within the tapered bore 15a of body potion. It is important to note that when the syringe cannula support 41 of the cannula assembly 26 is sealably connected to body portion 15 in the manner shown in FIG. 2, the syringe cannula or hypodermic needle 42 is stratigically positioned within lumen 18a of the adapter cannula 18 and is, therefore, protected from contamination, including contamination by the rubber stopper of the medicament container.

The syringe cannula support 41 of cannula assembly 26 is also provided with barrel connection means for connecting the assembly with the forward end 28 of the barrel portion 26 of the syringe body 25. This barrel connection means is here provided in the form of a conventional luer 48 formed on cannula support 41. As shown in the phantom lines of FIG. 3A, luer 48 is threadably receivable within internal threads 28a provided the forward end 28b of the barrel portion 28. Once the syringe cannula assembly 26 is interconnected with the syringe body 25, the syringe assembly 24 thus formed functions in the same manner as a conventional medicament administration syringe and, in the manner presently to be described, can be used to reconstitute lyophilized drugs and to administer medicaments to a patient in a conventional manner.

It is to be understood that the medicament container MC can contain a fluid medicament, or, alternatively, can contain a medicament in a powdered or lyophilized form. As previously mentioned, when the medicament is a powdered or lyophilized form a suitable liquid diluent must be added to the container to reconstitute the powdered or lyophilized drug before use. In accordance with one form of the method of the present invention, this can be accomplished by first accessing the sealed container of powdered or lyophilized drug using the vial accessing adapter means that is made up of adapter 14 and syringe cannula assembly 26. This accessing step is accomplished by placing the vial accessing adapter subassembly 12 over the medicament container MC and exerting a downward force on the subassembly sufficient to cause adapter cannula 18 to pierce the rubber stopper in the manner shown in FIG. 2A. With the components in the position shown in FIG. 2A, is to be observed that the lumen 18a of cannula 18 as well as the lumen 42a of piercing cannula 42 are in communication with the interior of medicament container MC.

With the vial accessing adapter subassembly 12 appropriately interconnected with the medicament container MC, the assemblage thus formed is inverted and a sealed syringe body 25 that has been prefilled with a suitable diluent opened and is then connected to the vial accessing adapter means by means of the luer connector 48 formed on member 41. The prefilled, sealed syringe body, which is typically readily available, off-the-shelf item, can be of various sizes and can contain various types of diluent. With the prefilled syringe body connected to the vial accessing adapter subassembly, a force exerted on plunger 30 will cause the diluent to controllably flow into the medicament container MC. The inverted medicament container is then shaken to thoroughly intermix the powdered or lyophilized drug with the liquid diluent. This done, the reconstituted drug can be drawn into the syringe assembly 24 by withdrawing the plunger 30 of the syringe body. The syringe assembly 24 and can then be removed from the adapter 14 and, the manner shown by the solid lines in FIG. 3A, the syringe can be used to administer the reconstituted drug to the patient. It is to be appreciated that throughout this entire process, cannula or hypodermic needle 42 has been maintained in a sterile configuration. Only cannula 18 has pierced the potentially contaminated rubber stopper RS of the medicament container and cannula 42 has been completely protected against any possible contamination by the rubber stopper RS. Stated another way, during the entire process of reconstituting the powdered or lyophilized drug, cannula 42 has been maintained in a virgin, sterile configuration and is completely free from any possible contamination at the time of administration of the reconstituted drug to the patient.

When the medicament to be delivered to the patient is contained within the medicament container and requires no reconstitution, the assembled syringe 24 can be mated with the adapter 14 and the assembly thus formed can be directly mated with the medicament container MC. In this instance, during the mating step, the skirt portion 20 of the adapter is snapped over the upper portion of the container and the cannula 18 is urged into piercing engagement the rubber stopper in the manner shown in FIG. 2A so as to open communication between lumen 42a of cannula 42 and the interior of the medicament container MC. The syringe assembly can then be used to withdraw the liquid medicament from the container into reservoir 32 of the syringe assembly. The syringe assembly can then be removed from the adapter 14 and used to inject the medicament within reservoir 32 into a patient in a conventional manner. It is to be understood that, if desired, the syringe cannula assembly can first be connected to the adapter 14 to form a container accessing subassembly comprising the syringe cannula assembly and the adapter 14. The container accessing subassembly can then be mated with the medicament container so that the cannula 18 pierces the rubber stopper. This done, the syringe body can be mated with the container accessing subassembly and by sliding the piston outwardly of the syringe body, the medicament can be removed from the container.

Turning next to FIGS. 4 and 4A, there is shown an alternate form of the vial accessing adapter means of the apparatus of the invention for the aseptic administration of medicaments contained within a medicament container. This alternate form of the invention is quite similar to the embodiment shown in FIGS. 1 through 3 and like numerals are used in FIGS. 4 and 4A to identify like components. The primary difference between this latest form of the invention and the earlier described embodiment resides in the fact that a filter means, shown here as a porous filter 50, is provided within lumen 18a of cannula 18. Filter 50, which can be constructed from any suitable porous metal or ceramic material, is strategically positioned between cannula 42 and the open-end 18a of cannula 18. When positioned within cannula 18, filter 50 functions to effectively filter out any particular matter that may reside within the liquid medicament contained within medicament container MC.

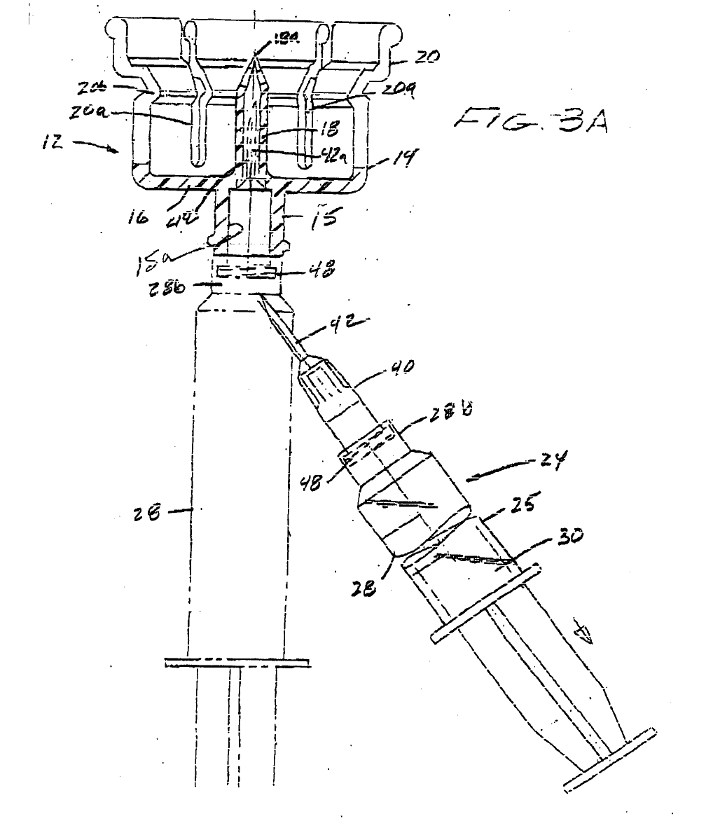

Referring next to FIGS. 5 and 5A, an alternate form of vial accessing adapter means of the invention is there shown in generally designated by the numeral 40. The vial accessing adapter means of this alternate form of the invention is somewhat similar to vial accessing adapter means of the embodiment of the invention shown in FIG. 1, but uniquely includes venting means for venting to atmosphere any gases that may reside within in the medicament container.

As shown in FIG. 5, this alternate form of vial accessing adapter means includes an adapter component 44 that is preferably formed from a moldable plastic material. Adapter 44 includes a body portion 45 having a tapered bore 45a and a top wall 46 connected to the body portion. An adapter cannula 48 is integrally formed with and extends from body portion 45. A container connector means is connected to top wall 46 for removably interconnecting the adapter component to a conventional medicament container MC, such as the container shown in FIG. 2. The container connector means of the present form of the invention here comprises a resiliently deformable skirt 50 that is integrally formed with and extends from top wall 46 in the manner shown in the drawings. When the adapter component is mated with the medicament container, skirt 50 telescopically receives and securely grips the upper portion of the medicament container in a manner such that adapter cannula 48 will completely pierce the rubber stopper of the container.

As before, plastic adapter cannula 48 has a piercing extremity 48a and a lumen 48b that communicates with the interior of the medicament container when the adapter is interconnected with the container in the manner previously discussed herein. In this regard, it is to be noted that as in the earlier described embodiments, skirt 50 is provided with a radially inwardly extending shoulder 50a and a plurality of circumferentially spaced slits 50b that enable the adapter of the invention to be snapped over the upper portion of the medicament container to securely grip the container in the manner shown in FIG. 2.

In this latest form of the invention cannula 48 is provided with a vent passageway 54 that also communicates with the interior of the medicament container. As best seen in FIG. 5A, vent passageway 54 communicates with a transversally extending passageway 56 via a filter member 58. Transversally extending passageway 56, in turn, communicates with a vent port 60 formed in top wall 46 of the adapter 44. The vent passageways 54 and 56, along with vent port 60 comprises the venting means of the form of the invention shown in FIGS. 5 and 5A. With the construction shown in these figure drawings, after plastic cannula 48 has pierced the rubber stopper of the medicament container, gases within the container can flow to atmosphere in the direction of the arrows shown in FIG. 5A and thereby affectively vent the interior of the container to atmosphere.

As before, body portion 45 functions to removably interconnect a syringe assembly of the character previously described and as shown in FIG. 3A. As in the earlier described embodiments of the invention, the tapered bore 45a of body portion 45 is adapted to sealably receive the syringe cannula support 41 of the syringe cannula assembly 26, or aspirator connector means, that comprises cannula support 41 and syringe cannula 42 that is connected to the syringe cannula support 41. Once the aspirating means or syringe body 25 is interconnected with the syringe cannula support 41 in the manner previously described, the syringe assembly thus formed functions in the same manner as a conventional medicament administration syringe to reconstitute lyophilized drugs and to administer medicaments to a patient in a conventional manner.

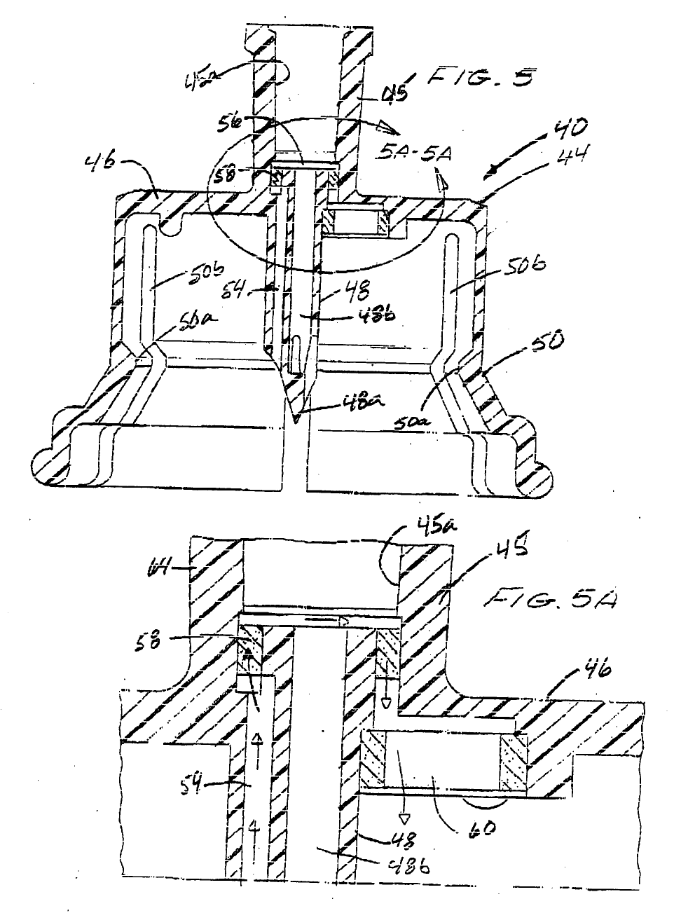

Turning to FIG. 6, still another, alternate form of the vial accessing adapter means of the invention is there shown in generally designated by the numeral 70. The vial-accessing adapter means of this alternate form of the invention is also somewhat similar to that shown in FIG. 1, but uniquely comprises an adapter 72 that includes generally cylindrically shaped connector skirt 74. Adapter 72 also includes a body portion 75 having a tapered bore 75a, a top wall 76 connected to the body portion and an adapter cannula 78 that is integrally formed with and extends from body portion 75. Connector skirt 74 is connected to top wall 46 and functions to removably interconnect the adapter component to a conventional medicament container such as the container MC shown in FIG. 2. When the adapter component is mated with the medicament container, skirt 74 telescopically receives and securely grips the upper portion of the medicament container in a manner such that adapter cannula 78 will completely pierce the rubber stopper of the container.

As before, plastic adapter cannula 78 has a piercing extremity 78a and a lumen 78b that communicates with the interior of the medicament container when the adapter is interconnected with the container in the manner previously discussed herein.

The tapered bore 75a of body portion 75 is adapted to removably receive the syringe cannula support 82 of the syringe cannula assembly that comprises cannula support 82 and syringe cannula 86 that is connected to the syringe cannula support 82. Once the syringe body 25 is interconnected with the syringe cannula support 82 in the manner previously described, the syringe assembly thus formed functions in the same manner as a conventional medicament administration syringe to reconstitute lyophilized drugs and to administer medicaments to a patient in a conventional manner.

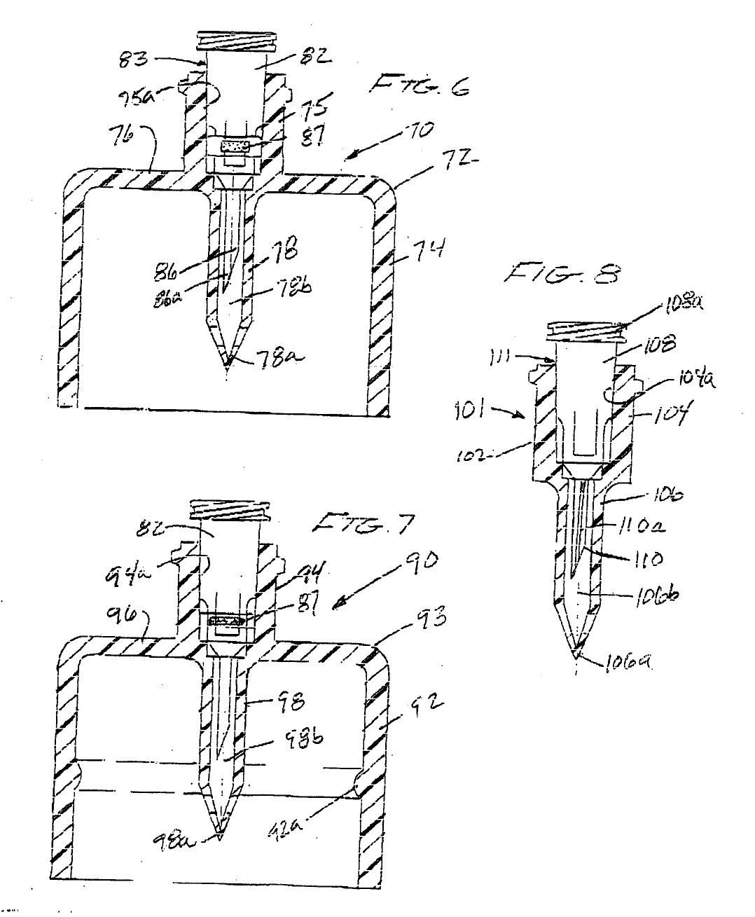

Referring next to FIG. 7, yet another, alternate form of the vial accessing adapter means of the invention is there shown in generally designated by the numeral 90. The vial accessing adapter means of this alternate form of the invention is quite similar to that shown in FIG. 6 and like numerals are used in FIG. 7 to identify like components. The principal difference between the adapter means of the invention shown in FIG. 7 and that shown in FIG. 6 resides in the fact that the generally cylindrically shaped plastic skirt 92 of the adapter 93 is provided with a circumferentially extending protuberance 92a. When the adapter of this alternate form of the invention is interconnected with the medicament container, protuberance 92a is lockably received proximate the neck of the medicament container MC and functions to hold the adapter in position relative to the medicament container.

As in the earlier described embodiments, adapter 93 includes a top wall 96 and an adapter cannula 98 that is integrally formed with and extends from top wall 96. Connector skirt 92 is also connected to top wall 96 and functions to removably interconnect the adapter component to a conventional medicament container such as the container shown in FIG. 2. When the adapter component is mated with the medicament container, skirt 92 telescopically receives the upper portion of the medicament container and protuberance 92a grips the neck of the container in a manner such that adapter cannula 98 completely pierces the rubber stopper of the container.

As before, plastic adapter cannula 98 has a piercing extremity 98a and a lumen 98b that communicates with the interior of the medicament container when the adapter is interconnected with the container in the manner previously discussed herein.

Adapter 93 includes a body portion 94 to which the top wall 96 is connected, the body portion having a tapered bore 94a. Cannula 98 is integrally formed with and extends from body portion 94 so that when the adapter component is mated with the medicament container, the adapter cannula pierces the stopper of the medicament container. Skirt 92 telescopically receives and securely grips the upper portion of the medicament container and as in the earlier described embodiments of the invention, the body portion 94 functions to sealably receive a syringe cannula support 82 which is identical in construction and operation to that previously described.

Turning lastly to FIG. 8, still another form of vial accessing adapter means of the invention for the aseptic administration of medicaments contained within a medicament container is there shown and generally designated by the numeral 101. This latest form of the invention includes an adapter component 102 that is somewhat similar to the adapter 14 shown in FIG. 1, but does not include either a top wall or a resiliently deformable skirt for gripping the medicament container. Rather, the adapter component 102 includes only a generally cylindrically shaped body portion 104 that is similar in configuration to the previously described adapter body portions. This generally cylindrically shaped body portion 104 of adapter 101 is provided with a tapered bore 104a that is adapted to sealably receive a syringe cannula support 108 to which a syringe cannula 110 of a syringe cannula assembly 111 is connected. Connected to and depending from body portion 104 is an adapter cannula 106 having a piercing extremity 106a that is adapted to pierce the rubber stopper of a conventional medicament container. As shown in FIG. 8, adapter cannula 106 has a lumen 106b that is in communication with bore 104a of body portion 104.

Once again, it is important to note that when the syringe cannula support 108 of the aspirator connector means is sealably received within tapered bore 104a of body portion 104 in the manner shown in FIG. 8, the syringe cannula or hypodermic needle 110 is once again strategically positioned within lumen 106b of the adapter cannula 106 and is therefore protected from contamination. Once the syringe cannula assembly 111 is interconnected with an aspirating means, such as the earlier described syringe body 25 (FIG. 3), the syringe assembly thus formed functions in the same manner as a conventional medicament administration syringe and can be used to reconstitute lyophilized drugs and to administer medicaments to a patient in the manner previously described herein.

In accordance with an alternate form of the method of the invention for reconstituting a powdered or lyophilized drug, the sealed container containing the drug is first accessed using the vial accessing adapter subassembly 101, which is of the character shown in FIG. 8. This accessing step is accomplished by exerting a force on the subassembly 101 that is sufficient to cause adapter cannula 106 to pierce the rubber stopper so that the lumen 106b of cannula 106 as well as the lumen 110a of cannula 110 are in communication with the interior of medicament container.

With the vial accessing adapter subassembly 101 appropriately interconnected with the medicament container, the assemblage thus formed is inverted and a sealed syringe body, such as syringe body 25 that has been prefilled with a suitable diluent is opened and is then connected to the vial accessing adapter subassembly 101 by means of the luer connector 108a formed on connector member 108. As before, the prefilled, sealed syringe body 25, or aspirator means, which is typically readily available, off-the-shelf item, can be of various sizes and can contain various types of diluent. With the prefilled syringe body connected to the vial accessing adapter subassembly 101, a force exerted on the plunger of the syringe will cause the diluent to controllably flow into the medicament container. The inverted medicament container is then shaken to thoroughly intermix the powdered or lyophilized drug with the liquid diluent. This done, the reconstituted drug can be aspirated into the syringe assembly by withdrawing the plunger of the syringe body. The syringe assembly can then be removed from body 104 and the syringe can be used to administer the reconstituted drug to the patient. It is to be appreciated that throughout this entire process, cannula or hypodermic needle 110 has been maintained in a sterile configuration. Only cannula 106 has pierced the rubber stopper of the medicament container and cannula 110 has been completely protected against any possible contamination by the rubber stopper of the medicament container.

When the medicament to be delivered to the patient is contained within the medicament container and requires no reconstitution, an assembled syringe, such as syringe 24 can be mated with body 104 of adapter 102 and the assembly thus formed can be directly mated with the medicament container. In this instance, during the mating step, the cannula 106 is urged into piercing engagement the rubber stopper so as to open communication between lumen 106b of cannula 106 and the interior of the medicament container. The syringe assembly can then be used to withdraw the liquid medicament from the container into the reservoir of the syringe assembly. The syringe assembly can then be removed from body 104 and used to inject the medicament within reservoir into a patient in a conventional manner.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following claims.

1. A vial accessing means for interconnecting an aspirator with a medicament container containing a medicament, comprising:
(a) an adapter including:
(i) a body portion having a bore;
(ii) an adapter cannula connected to and extending from said body portion, said adapter cannula having a lumen in communication with said bore of said body portion of said adapter; and
(ii) aspirator connector means connected to said body portion of said adapter for removably interconnecting the aspirator with said adapter, said aspirator connector means comprising a syringe cannula support and a syringe cannula connected to said syringe cannula support, said syringe cannula support being received within said bore of said body portion of said adapter in a manner to position said syringe cannula within said lumen of said adapter cannula.

2. The apparatus as defined in claim 1 in which said syringe cannula comprises a hypodermic needle having a piercing point.

3. The apparatus as defined in claim 1 in which said syringe cannula support includes a luer connector.

4. The apparatus as defined in claim 1 which said adapter further includes filter means for filtering particulate matter from medicament aspirated from the medicament container.

5. The apparatus as defined in claim 1 in which said adapter further includes container connector means connected to said body portion of said adapter, said container connector means comprising a resiliently deformable skirt connected to and extending from said body portion for receiving a portion of the medicament container.

6. The apparatus as defined in claim 5 which said adapter further includes vent means for venting to atmosphere gases contained within the medicament container.

7. An apparatus for the aseptic administration of medicaments contained within a medicament vial, comprising:
(a) aspirator means for aspirating medicaments from the medicament vial; and
(b) vial accessing adapter means for interconnecting said aspirator means with the medicament vial, said vial accessing adapter means comprising:
(i) an adapter including:
a. an adapter body portion having a bore;
b. an adapter cannula connected to and extending from said adapter body portion, said adapter cannula having a lumen in communication with said bore of said body portion; and
(ii) aspirator connector means for removably interconnecting said aspirator means with said adapter, said aspirator connector means comprising a syringe cannula support received within said bore of said adapter body portion and a syringe cannula connected to said syringe cannula support, said syringe cannula support being removably connected to said adapter body portion of said adapter in a manner such that said syringe cannula is disposed within said lumen of said adapter cannula.

8. The apparatus as defined in claim 7 in which said syringe cannula of said aspirator connector means comprises a needle having a piercing point.

9. The apparatus as defined in claim 7 in which said adapter cannula of said adapter comprises a plastic member having a piercing point.

10. The apparatus as defined in claim 7 in which said adapter further includes container connector means connected to said body portion, said container connector means comprising a skirt connected to and extending from said body portion for telescopically receiving a portion of the medicament container.

11. The apparatus as defined in claim 7 in which said aspirator means comprises an aspirator assembly including:
(a) a syringe body having a forward end; and
(b) a piston slidably carried within said syringe body to form a liquid chamber between said forward end of said syringe body and said piston, said syringe cannula being in communication with said liquid chamber when said syringe cannula support is removably connected to said adapter body portion.

12. The apparatus as defined in claim 11 in which said syringe cannula support of said aspirator connector means includes connection means for removably interconnecting said syringes cannula support of said aspirator connector means with said syringe body of said aspirator assembly.

13. A vial accessing adapter apparatus for interconnecting an aspirator with a medicament container containing a medicament, comprising:
(a) an adapter having:
(i) an adapter body portion having a tapered bore;
(ii) a top wall connected to said adapter body portion;
(iii) an adapter cannula connected to and extending from said adapter body portion, said adapter cannula having a lumen; and
(iv) a container connector means connected to said top wall of said adapter for removably interconnecting said adapter with the medicament container; and
(b) aspirator connector means connected to said adapter for removably interconnecting the aspirator with said adapter, said aspirator connector means comprising a syringe cannula support received within said bore of said adapter body portion and a syringe cannula connected to said syringe cannula support, said syringe cannula support being removably connected to said adapter body portion of said adapter in a manner such that said syringe cannula is disposed within said lumen of said adapter cannula.

14. The apparatus as defined in claim 13 in which said container connector means of said adapter comprises a resiliently deformable skirt connected to and extending from said top wall for telescopically receiving a portion of the medicament container.

15. The apparatus as defined in claim 13 in which said syringe cannula comprises a needle having a lumen and a piercing point.

16. The apparatus as defined in claim 13 in which said adapter cannula comprises a plastic member having a piercing point.

17. The apparatus as defined in claim 13 which said adapter further includes vent means for venting to atmosphere gases contained within the medicament container.

18. The apparatus as defined in claim 13 which said adapter further includes filter means for filtering particulate matter from medicament aspirated from the medicament container.

19. An apparatus for the aseptic administration of medicaments contained within a medicament container containing a medicament and having a top portion including a rubber stopper comprising:
(a) aspirator means, including:
(i) an aspirator body portion having a forward end, said aspirator body portion having internal threads;
(ii) a piston slidably carried within said aspirator body portion to form a liquid chamber between said forward end of said aspirator body portion and said piston; and
(iii) an aspirator connector connected to said aspirator body portion, said aspirator connector comprising a syringe cannula support and a syringe cannula connected to said syringe cannula support, said syringe cannula support having external threads mateably with said internal threads of said aspirator body portion; and
(b) adapter means for removably interconnecting said aspirator means with the medicament container, said adapter means comprising an adapter having:
(i) an adapter body portion having a bore for sealably receiving said syringe cannula support of said aspirator connector;
(ii) an adapter cannula connected to and extending from said adapter body portion, said adapter cannula having a lumen;
(iii) a top wall connected to said adapter body portion; and
(iv) a container connector means connected to said top wall of said adapter for removably interconnecting said adapter with the medicament container, said container connector means comprising a skirt connected to and extending from said top wall for telescopically receiving a portion of the medicament container.

20. The apparatus as defined in claim 19 in which said skirt is resiliently deformable and includes a plurality of circumferentially spaced slits.

21. The apparatus as defined in claim 19 further including filter means mounted within said adapter cannula for filtering out particulate matter contained within the medicament contained within the medicament container.

22. A method for the reconstitution of lyophilized drugs contained within a medicament container having a rubber stopper and for the direct injection of the reconstituted drug into a patient using an apparatus comprising an aspirator assembly that includes an aspirator body portion having a forward end, a piston slidably carried within the aspirator body portion to form a prefilled liquid chamber between the forward end of the aspirator body portion, the piston portion, and an aspirator connector comprising a cannula support and a hypodermic needle, the aspirator connector being removably connected to the forward end of the aspirator body portion in a manner such that the lumen of the hypodermic needle is in direct communication with the liquid chamber; and a cooperating adapter assembly that includes an adapter having an adapter body portion having a tapered bore for sealably receiving the cannula support, an adapter cannula having a lumen, the adapter cannula being connected to and extending from the adapter body portion, the method comprising the steps of:
(a) interconnecting the adapter assembly with the medicament container containing the lyophilized drug in a manner such that the adapter cannula pierces the rubber stopper of the medicament container and extends into the medicament container;
(b) interconnecting the cannula support of the aspirator connector with the adapter body portion in a manner such that the hypodermic needle of the adapter connector is received within the lumen of the adapter cannula;
(c) interconnecting the cannula support of the aspirator with the aspirator body;
(d) sliding the piston within the aspirator body in a manner to urge the liquid containing therewithin into the medicament container containing the lyophilized drug to intermix the liquid with the lyophilized drug to form a reconstituted medicament;
(e) sliding the piston within the aspirator body in a manner to withdraw the reconstituted medicament from the medicament container;
(f) removing the aspirator body and the aspirator connector from the adapter; and
(g) interconnecting the hypodermic needle with the patient and then sliding the piston within the aspirator body to force the reconstituted medicament contained within the aspirator body into the patient.

23. A method for the delivery to a patient of liquid medicaments contained within a medicament container having a rubber stopper using an apparatus comprising an aspirator assembly that includes an aspirator body portion having a forward end, a piston slidably carried within said aspirator body portion to form a liquid chamber between the forward end of said aspirator body portion; and an aspirator connector comprising a cannula support and a hypodermic needle, to aspirator connection being removably connected to the forward end of the aspirator body portion in a manner such that the lumen of the hypodermic needle is in direct communication with the liquid chamber; and a cooperating adapter assembly that includes an adapter having an adapter body portion having a tapered bore for sealably receiving the cannula support, a top wall connected to the adapter body portion, an adapter cannula having a lumen, the adapter cannula being connected to and extending from the adapter body portion; and a connector skirt connected to the top wall for removably interconnecting the adapter with the medicament container containing the medicament, the method comprising the steps of:
(a) interconnecting the adapter with the medicament container containing the liquid medicament in a manner such that the adapter cannula pierces the rubber stopper of the medicament container and extends into the medicament container;
(b) interconnecting the aspirator connector with the adapter body portion of the adapter in a manner such that the hypodermic needle of the aspirator connector assembly is received within the lumen of the adapter cannula;
(c) interconnecting the aspirator connector with the adapter body of the aspirator assembly;
(d) sliding the piston within the adapter body portion in a manner to withdraw the liquid medicament from the medicament container;
(e) removing the adapter body and the aspirator connector that is interconnected therewith from the adapter body; and
(f) interconnecting the hypodermic needle with the patient and then sliding the piston within the syringe body to force the liquid medicament contained there within into the patient.

24. A method for the delivery to a patient of a liquid medicament contained within a medicament container having a rubber stopper using an apparatus comprising an aspirator assembly that includes an aspirator body portion having a forward end, a piston slidably carried within said aspirator body portion to form a liquid chamber between the forward end of said aspirator body portion; the piston and an aspirator connector comprising a cannula support and a hypodermic needle that can be removably connected to the forward end of the aspirator body portion in a manner such that the lumen of the hypodermic needle is in direct communication with the liquid chamber; and a cooperating adapter assembly that includes an adapter having an adapter body portion having a tapered bore for sealably receiving the cannula support, a top wall connected to the adapter body portion, an adapter cannula having a lumen, the adapter cannula being connected to and extending from the adapter body portion, and a skirt connected to the top wall for removably interconnecting the adapter with the medicament container containing the medicament, the method comprising the steps of:
(a) interconnecting the aspirator body portion with the aspirator connector to form a container accessing subassembly;
(b) interconnecting the adapter with the medicament container containing the liquid medicament in a manner such that the adapter cannula of the adapter pierces the rubber stopper of the medicament container;
(c) interconnecting the container accessing subassembly with the adapter;
(d) sliding the piston within the adapter body in a manner to withdraw the liquid medicament from the medicament container;
(e) removing the container accessing subassembly from the adapter; and
(f) interconnecting the hypodermic needle with the patient and then sliding the piston within the adapter body to force the medicament contained within the adapter body into the patient.
